(19) United States
(12) Patent Application Publication
(10) Pub. No.: US 2006/0161134 A1
(43) Pub. Date: Jul. 20, 2006

(54) Intravenous connector device

(76) Inventor: Timothy Michael Craft, Tach (GB)

Correspondence Address: Vincent L. Ramik;DILLER, RAMIK & WIGHT, Suite 101, 7345 McWhorter Place, Annandale, VA, 22003, US

(21) Appl. No.: 11/037,217

(22) Filed: Jan. 19, 2005

(51) Int. Cl. A61M 25/00

(52) U.S. Cl. 604/523; 604/43

(57) ABSTRACT

An intravenous connector device for coupling a plurality of intravenous fluid conduits to a single intravenously insertable portion. The device includes a body having two or more inlet lumens for receiving fluid conduits in which at least one of the lumens has a mechanism for limiting the depth to which a fluid conduit can be inserted in use.

### BACKGROUND OF THE INVENTION

The present invention relates generally to an intravenous connector device and particularly to a connector device for coupling a plurality of intravenous fluid conduits to a single intravenously insertable portion such as a cannula or catheter.

In many cases it is desirable to administer several different fluids to a patient at the same time. For example, it is desirable to administer a separate fluid infusion with a drug infusion as this helps to flush the drug into the patients venous system and also helps to keep the vein open during drug infusions; this also helps to reduce the chance of an adverse reaction to the drug.

In order to infuse fluids into a vein, devices such as a cannula or catheter are used as an intravenously insertable portion. The ability to infuse a plurality of different fluids through a single insertable portion is advantageous because only a single puncture is required.

Whilst multiple fluid conduit connectors are known they do not control the depth to which conduits are inserted into the device and this can lead to problems such as different tubes being inserted to a different extent into the device and tubes not being inserted sufficiently into the device and so not being secured in position.

The present invention seeks to address the problems with known intravenous connector devices.

### SUMMARY OF THE INVENTION

According to the present invention there is provided an intravenous connector device for coupling a plurality of intravenous fluid conduits to a single intravenously insertable portion, the device comprising a body having two or more inlet lumens for receiving fluid conduits, in which at least one of the lumens has means for limiting the depth to which a fluid conduit can be inserted in use.

The connector device of the present invention therefore includes lumens which have a defined point to which fluid conduits can be inserted. Accordingly, all fluid conduits in the connector device can be connected to a required depth and a user will feel an increased resistance once the conduits have been inserted to the required extent.

The conduits may be insertable so as to be removable; alternatively more permanent attachment means may be employed. For example, the conduits may be attached into the lumens by medical glue by first coating the exterior of the conduit end with glue and then inserting the conduit into the lumen so that adhesion is achieved between the exterior wall of the conduit and the interior wall of the lumen.

The device may further comprise attachment means for attaching it to an insertable portion. For example, the device may be provided with a Luer lock system in which a male or female Luer lock component is provided at the end of the device for attaching to a female or male Luer lock component on a cannula, catheter or the like.

The depth limiting means may comprise a formation in the or each lumen. The formation may be formed, for example, as a shoulder or continuous annulus or may comprise one or more individual projections which project radially inwardly from the lumen walls. A shoulder portion defining a radially inward region may be preferred because of the ease of moulding and de-moulding a structure which has an absence of undercuts.

At least one of the lumens may taper inwardly towards its distal end remote from the end at which the fluid conduit is inserted. Tapering lumens have the advantage that the lumen can be slightly larger than required at its proximal end to facilitate initial insertion and then by the gradual reduction in lumen diameter the tube can be forced towards the distal end and held tightly in place.

The device may have three inlet lumens and thus constitute a trifurcated hub-type device. Where three lumens are provided they may be arranged in a trilobate, “clover leaf” configuration in order to give the most compact arrangement of lumens.

The inlet lumens may have different diameters to each other. This would enable fluid conduits of different diameters to be used in the same device.

The space between the point at which the fluids from the lumens enter a common volume may be minimised. This is particularly advantageous where fluids being coupled have physical and/or pharmacological incompatibility so that they do not have chance to mix before reaching the patients blood stream.

The device may comprise a single common final outlook portion in fluid communication with the inlet lumens. Therefore the device may have a “Y” configuration in which a common final length is provided prior to the distal end of the device which connects to the insertable portion. This is opposed to a device of a “V” configuration in which each of the lumens terminate at the distal end of the device, which is also possible in the case of the present invention.

The device may be formed from a plastics material.

The device may be formed by a moulding process, such as injection moulding.

The present invention may also provide an intravenous fluid infusion system comprising a connector device as described herein together with fluid conduits connected in the lumens.

The present invention will now be more particularly described, by way of example, with reference to the accompanying drawings, in which:

### BRIEF DESCRIPTION OF THE DRAWINGS

FIG. 1 is a side view of a connector device formed according to the present invention.

FIG. 2 is a plan view of the proximal end of the connector of FIG. 1.

FIG. 3 is a section of the device of FIG. 1.

FIG. 4 is a side view of a connector device formed according to an alternative embodiment.

FIG. 5 is a section of the device of FIG. 4 along lines V-V.

FIG. 6 is a plan view of the proximal end face of the device of FIGS. 4 and 5.

FIG. 7 is a section of a connector device formed according to an alternative embodiment.

FIG. 8 is a plan view of the proximal end of the connector device of FIG. 7.

FIG. 9 is a plan view of the proximal end of a connector device formed according to an alternative embodiment.

FIG. 10 shows an intravenous fluid infusion system formed according to the present invention.

### DETAILED DESCRIPTION OF THE PREFERRED EMBODIMENTS

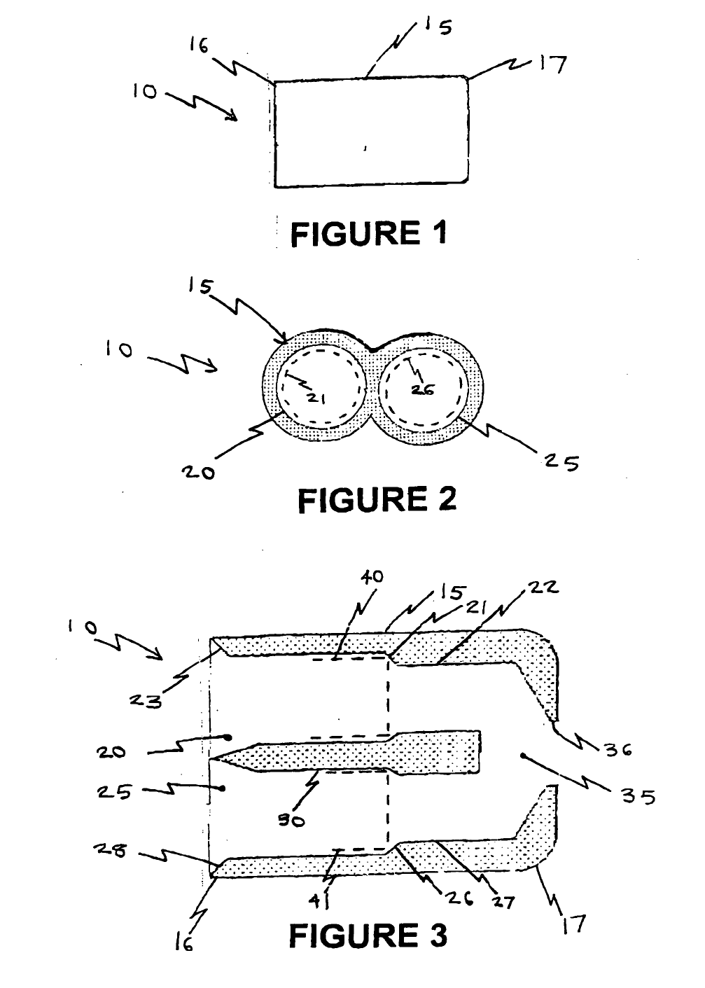

Referring first to FIGS. 1 to 3 there is shown a connector device generally indicated 10. The device 10 comprises a generally cylindrical body 15 having a proximal end 16 and a distal end 17.

At the proximal end 16 two inlet lumens 20, 25 extend distally. The lumens 20, 25 are separated by a central stem 30 giving the distal end 16 a generally “figure of eight” configuration (see FIG. 2).

Approximately half way along the length of the body 15 each of the lumens 20, 25 reduces in diameter at shoulders 21, 26.

Distal to the shoulders 21, 26 the lumens 20, 25 continue with outlet linking portions 22, 27 of reduced diameter. The linking portions 22, 27 are also separated by the central stem 30.

The linking portions 22, 27 lead to a common outlet chamber 35 which leads to an outlet 36.

In use, intravenous fluid supply tubes 40, 41 (their distal ends are shown in phantom) can be inserted into the lumens 20, 25. The insertion is facilitated by tapering openings 23, 28 at the proximal ends of the outlets 20, 25. Fluid tubes 40, 41 are pushed into the lumens 20, 25 until they reach the shoulders 21, 26 at which point the diameter restriction prevents further insertion. The shoulders 21, 26 thereby constitute depth limiting means. In addition the shoulders provide the tubing with a firm seating.

Fluid is supplied through the tubes 40, 41 and the fluid flows via the linking portions 22, 27 into the common chamber 35 and then out of the outlet 36. The outlet 36 is fluidly connected in some way to an insertable portion such as a cannula or a catheter (not shown) so that fluids from both of the tubes 40, 41 can be coupled into a single insertable portion via the single common outlet 36.

The space between the point at which the fluids from the lumens 20, 25 enter the common volume 35 is minimised. This is advantageous where fluids being coupled have physical and/or pharmacological incompatibility so that they do not have chance to mix before reaching the patients blood stream.

The tubes 40, 41 may be secured in position within the lumens 20, 25 by the use of medical glue which is applied to the exterior surface of the distal ends of the tubes 40, 41 before inserting them into the lumens so that the exterior walls of the tubes 40, 41 can bond to the interior walls of the lumens 20, 25.

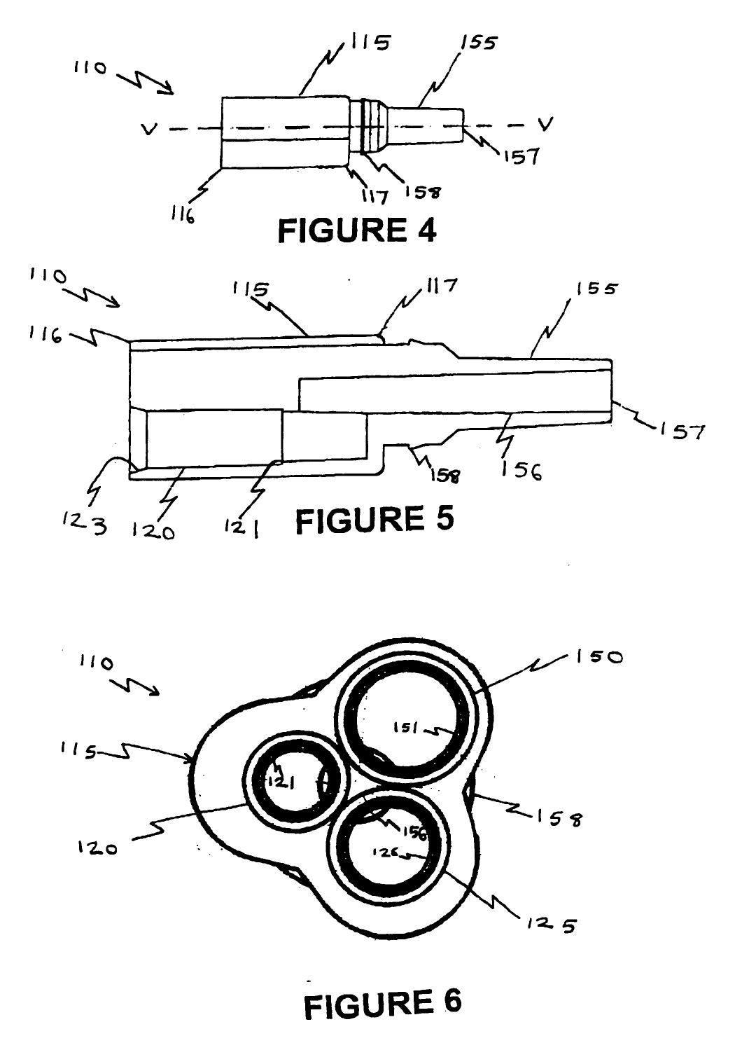

Referring now to FIG. 4 to 6 there is shown a device 110 according to an alternative embodiment. The device 110 comprises a body 115 and is in many ways similar to the device 10 of FIGS. 1 to 3. However, in this embodiment the device 110 includes three lumens 120, 125, 150. The lumens 120, 125, 150 have different diameters, with the lumen 150 having the largest diameter, and the lumen 120 having the smallest diameter.

The body 115 has a generally trilobate configuration with one lumen 120, 125, 150 provided in each lobe in order to minimise the dimensions of the body 115.

The lumens 120, 125, 150 taper inwardly from their proximal ends and this allows a tube to be forced into the lumens and gripped tightly.

Each of the lumens 120, 125, 150 has a diameter restriction in the form of a shoulder 121, 126, 151. The shoulders 121, 126, 151 function in the same way as the shoulders 21, 26 of the device 10 shown in FIGS. 1 to 3 to limit the depth to which tubes can be inserted.

In this embodiment the device 110 includes attachment means in the form of a male Luer probe 155 which extends from the distal end 117 of the body 115. The probe 155 includes a single central outlet conduit 156 into which each of the lumens 120, 125, 150 empties. At the distal end of the conduit 156 is an outlet 157.

The probe 155 includes a shoulder portion 158 which comprises a male collar which can be used to connect a cannula or catheter via a corresponding female collar (not shown).

Use of the device 110 is generally the same as that described in relation to FIGS. 1 to 3 in that tubes (not shown) are inserted into the lumens 120, 125, 150 to a depth defined by the shoulders, 121, 126, 151. Thereafter when fluids are run through the tubes the fluids empty into the common outlet conduit 156 and can then be infused into a patient via the outlet 157 which connects to an insertable portion.

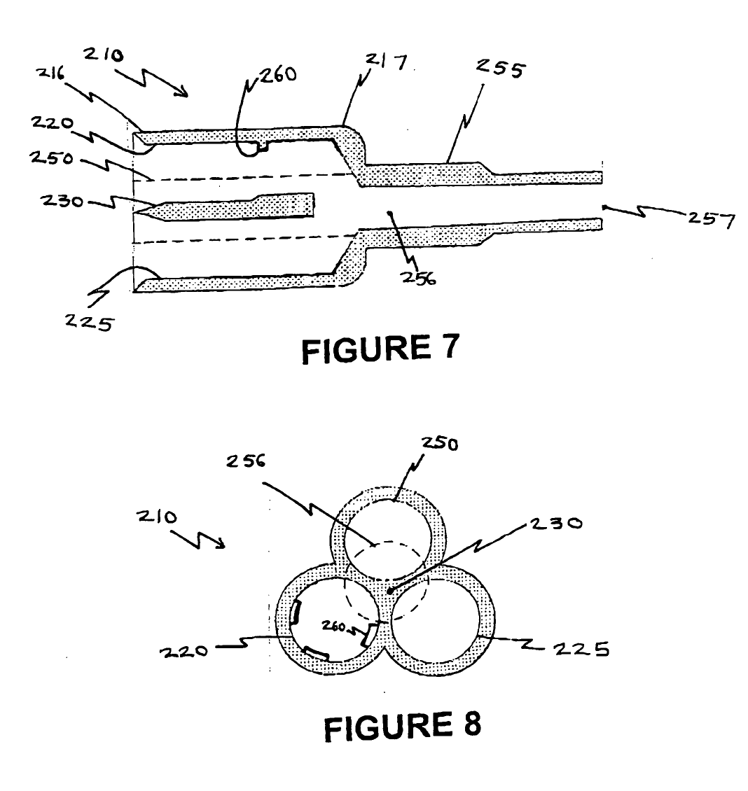

Referring now to FIGS. 7 and 8 there is shown a device 210 according to an alternative embodiment. The device 210 is similar to the device 110 shown in FIGS. 4 to 6 in that a body 215 is provided with three lumens 220, 225, 250 in a trilobate configuration. Each of the lumens 220, 225, 250 is substantially circular in section and in this embodiment are all of substantially the same diameter.

At the distal end 217 of the body 215 is an attachment portion 255 which serves as the connection point for a cannula, catheter or the like. The attachment portion 255 includes a single common outlet conduit 256 leading to an outlet 257.

Depth limiting means for liming the extent to which a tube can be inserted are provided. In this embodiment only the lumen 220 is provided with depth limiting means, which are in the form of three projections at 260 extending radially inwardly from the wall of the lumen 220.

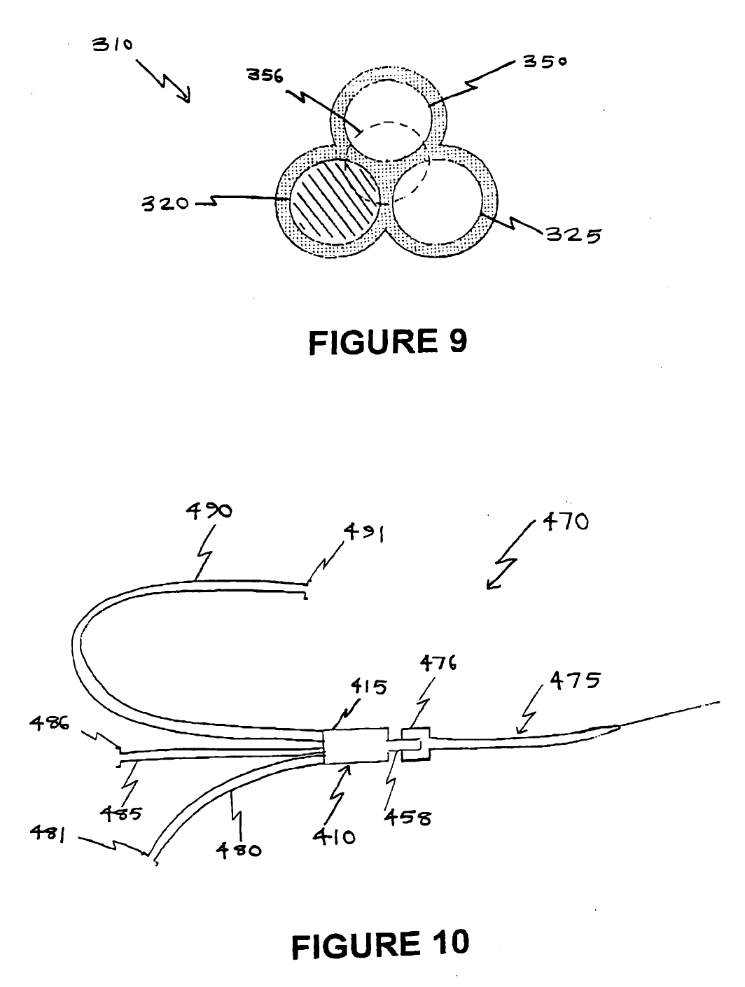

Referring now to FIG. 9 there is shown a device 310 according to an alternative embodiment. The device 310 is similar to the device 210 shown in FIGS. 7 and 8. A body 315 with a trilobate configuration is provided. However, in this embodiment only two active lumens 325, 350 are provided. It will be noted that the equivalent of the lumen 220 shown in FIG. 8 is not present. The lobe of the body 315 is blocked so that only two active lumens 325, 350 are provided in a trilobate body. This could be achieved in an injection moulding operation by including a blank in the mould to block the lumen 320. In this way, the trilobate body 315 could be used as a bi-lumen device without requiring extensive modification.

Referring now to FIG. 10 there is shown an intravenous fluid infusion system generally indicated 470. The system 470 comprises a connector device 410 of the general type described in relation to FIGS. 1 to 9. The device 410 is connected to an insertable portion 475 in the form of a catheter. The catheter 475 is connected to the body 410 via a Luer lock system with a female Luer lock component 476 provided at the proximal end of the catheter 475 and a male Luer lock component 458 provided at the distal end of the body 415 of the device 410.

The device 410 receives three infusion lines 480, 485, 490 having respective attachment components 481, 486, 491 for connecting the lines to infusion fluid sources (not shown) for supplying fluids such as drugs in solution.

The lines 480, 485 are fine polyethylene-lined extension lines. The line 490 comprises a large bore, semi-rigid line. The line 490 is pre-formed into a C-shape and is kink-resistant.

In use the device 410 can be used to couple fluids arriving separately from the lines 480, 485, 490 into the common catheter 475.

1. An intravenous connector device for coupling a plurality of intravenous fluid conduits to a single intravenously insertable portion, the device comprising a body having two or more inlet lumens for receiving fluid conduits, in which at least one of the lumens has means for limiting the depth to which a fluid conduit can be inserted in use.

2. The device according to claim 1, the device further comprising attachment means for attaching the device to an insertable portion.

3. The device according to claim 1, in which the limiting means comprise a formation in the or each lumen.

4. The device according to claim 1, in which the limiting means comprise a shoulder portion.

5. The device according to claim 1, in which at least one of the lumens tapers inwardly towards its distal end.

6. The device according to claim 2, in which the attachment means comprise a male or female Luer lock component.

7. The device according to claim 1, in which the device has three inlet lumens.

8. The device according to claim 7, in which the lumens are arranged in a trilobate configuration.

9. The device according to claim 1, in which the inlet lumens have different diameters to each other.

10. The device according to claim 1, in which the device comprises a single common final outlet portion in fluid communication with the inlet lumens.

11. An intravenous fluid infusion system comprising a connector device according to claim 1 having fluid conduits connected in the lumens.

12. (canceled)

13. (canceled)

14. The device according to claim 2, in which the limiting means comprise a formation in the or each lumen.
